SEQUENCE LISTING (10 sequences)

SEQ ID NO: 1 - DNA, 35 bp, Artificial
  Artificially generated exemplary nucleotide sequence
gctactactagctactactagctactactagtcta

SEQ ID NO: 2 - DNA, 17 bp, Artificial
  Artificially generated primer
gtaaaacgacggccagt

SEQ ID NO: 3 - DNA, 19 bp, Artificial
  Artificially generated exemplary nucleotide sequence
gtaaaacgacggccagtgc

SEQ ID NO: 4 - DNA, 20 bp, Artificial
  Artificially generated exemplary nucleotide sequence
gtaaaacgacggccagtgcc

SEQ ID NO: 5 - DNA, 26 bp, Artificial
  Artificially generated exemplary nucleotide sequence
gtaaaacgacggccagtgccatagac

SEQ ID NO: 6 - DNA, 36 bp, Artificial
  Artificially generated exemplary nucleotide sequence
gtaaaacgacggccagtgccatagactagtagtagc

SEQ ID NO: 7 - DNA, 46 bp, Artificial
  Artificially generated exemplary nucleotide sequence
gtaaaacgacggccagtgccatagactagtagtagctagtagtagc

SEQ ID NO: 8 - DNA, 56 bp, Artificial
  Artificially generated exemplary nucleotide sequence
gtaaaacgacggccagtgccatagactagtagtagctagtagtagctagtagtagc

SEQ ID NO: 9 - DNA, 19 bp, Artificial
  Artificially generated exemplary nucleotide sequence
actttcactttcaccccca

SEQ ID NO: 10 - DNA, 19 bp, Artificial
  Artificially generated exemplary nucleotide sequence
acccccacccccaccccca

(19) United States
(12) Patent Application Publication
(10) Pub. No.: US 2005/0095610 A1
(43) Pub. Date: May 5, 2005

(54) Method of generating size standard nucleic acids

(76) Inventors: Sophia S. Kuo, San Francisco, CA (US); Chu-an Chang, Piedmont, CA (US); Shiaw-Min Chen, Fremont, CA (US); Sandra L. Spurgeon, San Mateo, CA (US)

Correspondence Address: Donald R. Holland;Harness, Dickey & Pierce, P.L.C., 7700 Bonhomme, Suite 400, St. Louis, MO, 63105, US

(21) Appl. No.: 10/819,657

(22) Filed: Apr. 7, 2004

Related U.S. Application Data
(60) Provisional application No. 60/462,281, filed on Apr. 11, 2003.

(51) Int. Cl. C12Q 1/68; C12P 19/34

(52) U.S. Cl. 435/6; 435/91.2

(57) ABSTRACT

Methods for generating nucleic acid size standards are disclosed. The methods comprise providing a template polynucleotide which comprises periodic sequences of from about 5 to about 50 contiguous nucleotides containing not more than three types of nucleotides and wherein adjacent periodic sequences are separated by a terminator complement nucleotide that differs from the not more than three types of nucleotides, and performing a primer extension reaction on the template polynucleotide in the presence of nucleoside triphosphate molecules and a 3′ terminating nucleoside triphosphate which is complementary to the terminator complement nucleotide.

### CROSS-REFERENCE TO RELATED APPLICATIONS

This application claims the benefit of U.S. Provisional Application No. 60/462,281, filed on Apr. 11, 2003, which is hereby incorporated in its entirety by reference.

### FIELD

The present invention relates to molecular biology, and, in particular, to nucleic acid size standards and methods therefor.

### BACKGROUND

Nucleic acid research nearly always involves evaluation of the length of the nucleic acid molecules under investigation. This evaluation depends upon the use of nucleic acid size-standards. A number of methods have been used to generate size standards. Some of such methods include the generation of a set of concatenated nucleic acid molecules to form a ladder (see for example Zhang et al, Electrophoresis 14: 290-295, 1993; Diegelman et al., BioTechniques 25: 754-758, 1998; Louie et al., Nucleic Acids Research 18: 3090, 1990), the use of restriction enzyme digestion of a vector of known sequence to form a series of fragments of known size (see for example, Cooney, Mol. Biotechnol. 2: 119-127, 1994); Schwartz, Trends Genet. 12: 397, 1996) and the generation of specific fragments of a known sequence using PCR amplification of known portions of the sequence (Brondani et al, BioTechniques 31: 793-800, 2001). Some commercially available size standards made by such methods can, however, show anomalous migration times due to conformational effects which are related to the nucleic acid sequence (see for example Atha, Electrophoresis 19: 1428-1435, 1998). It would therefore be advantageous for researchers to have available nucleic acid size standards which are of a known length and sequence.

### SUMMARY

Accordingly, the inventors herein have succeeded in devising a method for generating nucleic acid size standards in which the nucleic acid size standards are of known length and sequence. In various embodiments, the method comprises providing a template polynucleotide having a sequence which contains not more than three different types of nucleotide bases, and a terminator complement type of nucleotide base different from each of the not more than three different types of nucleotide bases. The terminator complement type of nucleotide is present only at sites spaced periodically along the sequence. In various embodiments, the periodically spaced terminator complement type nucleotide is spaced evenly along the sequence. The template polynucleotide, in various embodiments, is an artificial sequence. In some embodiments, the sequence does not occur in nature. The method further involves performing a polymerase extension reaction in the presence of a 3′ terminating nucleotide complementary to the periodically spaced terminator complement type nucleotide. Nucleic acid fragments of various lengths are thus produced as a result of the different fragments terminating at the sites of the periodically spaced terminator complement type nucleotide along the template polynucleotide.

Thus, in various embodiments, there is provided a method for generating nucleic acid size standards. The method comprises combining in a mixture, a template polynucleotide having a 5′ portion and a 3′ template portion, a primer that is sufficiently complementary to the polynucleotide to hybridize therewith, a nucleic acid polymerase, nucleoside triphosphate molecules suitable for a polymerase extension of the primer on the template polynucleotide and a 3′ terminating nucleoside triphosphate. In some embodiments, the primer is sufficiently complementary to 3′ portion of the polynucleotide to hybridize therewith. The nucleoside triphosphate molecules can be, in some embodiments, deoxyribonucleosides such as one or more of deoxyadenosine triphosphate, deoxycytodine triphosphate, deoxyguanosine triphosphate or deoxythymidine triphosphate or the nucleoside triphosphate molecules can be ribonucleosides such as the corresponding ribonucleoside triphosphates. The 3′ terminating nucleoside triphosphate can be any of a number of nucleoside triphosphates that can be added to the 3′ end of an extension polynucleotide during a polymerase extension reaction, but inhibit further 3′ extension, for example a dideoxynucleoside triphosphate or an 3′ amino-substituted sugar moiety of a deoxyribonucleotide triphosphate. The method further comprises maintaining the mixture under conditions suitable for a primer extension reaction. The template polynucleotide comprises periodic sequences of from about 5 to about 50 contiguous nucleotides none of which are complements to the 3′ terminating nucleoside triphosphate. In addition adjacent periodic sequences are separated by the complementary nucleotide of the 3′ terminating nucleoside triphosphate.

In various embodiments, the present invention also includes methods for generating nucleic acid size standards involving providing a template polynucleotide comprised of periodic sequences. The periodic sequences can be from about 5 to about 50 contiguous nucleotides in length. Such periodic sequences can contain not more than three different types of nucleotides and the template polynucleotide sequence is such that adjacent periodic sequences are separated by a terminator complement type of nucleotide different from each of the not more than three different types of nucleotides. The method further involves performing a primer extension reaction on the template polynucleotide. The reaction utilizes a primer which is sufficiently complementary to the template polynucleotide to hybridize therewith, a nucleic acid polymerase, nucleoside triphosphate molecules suitable for a polymerase extension of the primer on the template polynucleotide and a 3′ terminating nucleoside triphosphate which is complementary to the terminator complement type of nucleotide.

In various embodiments, it can be advantageous for the periodic sequences to be substantially of the same length, which can be, for example, about 10 nucleotides, about 15 nucleotides, about 20 nucleotides, about 25 nucleotides, about 30 nucleotides, or about 50 nucleotides in length. In some embodiments, the periodic sequences can contain from about 6 to about 20 contiguous nucleotides. In some embodiments, the periodic sequences can be random sequences of not more than three different types of nucleotides other than the terminator complement type of nucleotide. In various embodiments, a periodically spaced terminator complement type of nucleotide can be spaced evenly along the template between the periodic sequences, for example every 10^{th} position, every 20^{th} position, every 30^{th} position, every 40^{th} position or every 50^{th} position.

In various embodiments, a template polynucleotide can be at least about 200 nucleotides in length, at least about 500 nucleotides in length, or at least about 1000 nucleotides in length, and the method can be used to generate size standards which can range in size from at least about 10 contiguous nucleotides or about 25 contiguous nucleotides in length to at least about 200 contiguous nucleotides in length, to at least about 500 contiguous nucleotides in length, or to at least about contiguous 1000 nucleotides in length. In various embodiments, the method can be used to generate size standards ranging from 500 contiguous nucleotides to 1000 contiguous nucleotides in length. In various embodiments, implementation of the method can generate at least about 10 size standard polynucleotides, at least about 20 size standard polynucleotides, at least about 30 size standard polynucleotides, at least about 40 size standard polynucleotides, or at least about 50 size standard polynucleotides.

In various embodiments, the method generates size standards which can be from about 25 contiguous nucleotides in length to about 1000 contiguous nucleotides in length. In a ladder generated using the method, fragments can differ in length by a pre-selected amount, for example, 6 nucleotides, 10 nucleotides, 14 nucleotides, 20 nucleotides, 25 nucleotides, or 50 nucleotides, or by combinations of pre-selected length differences.

In various embodiments, the sequence of a template polynucleotide can include one or more periodic sequences containing one or more “landmark nucleotides” which are complementary to a terminator that can be located at sites spaced differently compared to the majority of the periodically spaced terminator complements in the sequence. A nucleic acid fragment including such a sequence can act as a “landmark nucleic acid” when synthesized using the template polynucleotide as template and detected following size separation. A landmark nucleic acid, in which the size difference between adjacent periodic nucleic acids differs from regularly spaced periodic nucleic acid size standards, can facilitate identification of individual size standards comprising the ladder. For example, in some embodiments, the landmark nucleotides can be a triplet of identical bases complementary to a terminator and landmark nucleic acids can appear as a triplet of closely spaced nucleic acid fragments among evenly spaced nucleic acid fragments of greater size difference.

In various embodiments, the method can include using a detectable label that can be covalently attached to or incorporated into a template polynucleotide, a nucleoside triphosphate suitable for a polymerase-catalyzed extension of the primer, or a 3′ terminating nucleoside triphosphate. The label can be, for example, a fluorophore, a chromophore, a biotin, a hapten, a radioisotope, a chemiluminescent moiety, or a spin label. In various embodiments, the label can be a fluorophore. In various embodiments, the label can be a fluorophore which can be covalently attached to a 3′ terminating nucleoside triphosphate. The fluorophore can be any fluorophore that can be covalently attached to a nucleic acid without causing substantial anomalies in a nucleic acid's electrophoretic mobility.

### BRIEF DESCRIPTION OF THE DRAWING

FIG. 1 is an image of simulated nucleic acid size standards as they might appear if separated electrophoretically using capillary electrophoresis.

### DETAILED DESCRIPTION

Unless otherwise indicated, molecular biology methods known in the art are used (see Sambrook, J., et al., Molecular Cloning: A Laboratory Manual; Cold Spring Harbor Laboratory: Plainview, N.Y., 1989) The following definitions are used in describing the various embodiments disclosed herein.

The term “denaturation” as used herein refers to separation of the strands of a fully or partially double-stranded nucleic acid. A denaturation of a double-stranded nucleic acid can be effected by any means known in the art, such as (but not limited to) heating the double-stranded nucleic acid.

The term “high stringency hybridization” as used herein refers to high stringency conditions for hybridization as set forth in Sambrook et al., Molecular Cloning: A Laboratory Manual; Cold Spring Harbor Laboratory: Plainview, N.Y., 1989.

The term “hybridization” as used herein refers to formation of a double stranded nucleic acid comprising at least two single-stranded nucleic acids. The double-stranded structure can be completely double-stranded or partially double-stranded.

The term “oligonucleotide” as used herein refers to a polymer that can serve as a template for nucleic acid synthesis catalyzed by a polymerase. In some embodiments, a nucleotide subunit of an oligonucleotide can comprise a nucleotide base that can form a base pair with another nucleotide base, in non-limiting example adenine, thymine, cytosine, guanine, uracil, 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, beta, D-galactosylqueuosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, beta, D-mannosylqueuosine, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, pseudouridine, queuosine, 5-methyl-2-thiouridine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine, and 3-(3-amino-3-carboxy-propyl)uridine. In some embodiments, a nucleotide subunit of an oligonucleotide can further comprise a sugar, for example a five-carbon sugar such as a ribose, a deoxyribose, or a dideoxyribose, or a derivative thereof. In some embodiments, a nucleotide subunit of an oligonucleotide can further comprise a moiety that can link a sugar to another sugar, for example a phosphate or a sulphate.

In some embodiments, a “type” of nucleotide refers to the species of nucleotide that can serve as base-pairing partners to a nucleotide in a naturally-occurring DNA or mRNA. For example, a nucleotide which can form a base pair with an adenosine can be an “adenosine-pairing” type of nucleotide. Non-limiting examples of adenosine-pairing type nucleotides are thymidine, uridine, and 5-bromouridine. In some embodiments, there are no more than four different “types” of nucleotides, irrespective of the number of nucleotide species.

The term “terminator” as used herein refers to a nucleoside triphosphate which, if used in the elongation of a polynucleotide, inhibits further addition of a subsequent nucleotide or nucleotide analog to the 3′ terminal by a polymerase. Non-limiting examples of terminators are disclosed in Sanger et al., Proc. Natl. Acad. Sci. USA 76: 5463-5467, 1977; Hobbs et al., U.S. Pat. No. 5,047,519; Martinez et al., Nucleic Acid Research 27: 1271-1274, 1999; Metzger et al., Nucleic Acid Research 22: 4259-4267, 1994; Yuzhakov et al., FEBS Lett. 306: 185-188 1992; Dyatkina et al., FEBS Lett. 219:151-155, 1987; Chidgeavadze et al., Biochim. Biophys. Acta 868: 145-152, 1986; Chidgeavadze et al.; FEBS Lett. 183: 275-278, 1985; and Chidgeavadze et al., Nucleic Acids Research 12: 1671-1686, 1984). Non-limiting examples of terminators are a 3′-nucleoside triphosphate wherein the sugar can be a pentose (for example, a ribose or a deoxyribose) substituted at the 3′ carbon, wherein the 3′ substituent can be a hydrogen, an amino, an alkylamino, a halogen, a mercaptan, an alkoxy, or an aryloxy.

The term “terminator complement” type of nucleotide as used herein can be a type of nucleotide complementary to a species of nucleotide comprising a terminator nucleoside triphosphate.

In various embodiments, the inventors of the present application have developed methods of producing single-stranded nucleic acid size standards. In various embodiments, a collection of nucleic acid size standards produced using a method comprises a “ladder,” i.e., a collection of nucleic acids which vary in size by pre-selected intervals. In some embodiments, the polynucleotides of the invention appear nearly evenly spaced when imaged after separation according to size.

Various embodiments of the invention include methods for generating a set of size standard nucleic acids. In these embodiments, a polynucleotide of the invention is used as a template for generation of a nested set of size standard nucleic acids. In these embodiments, the method comprises contacting in a mixture, a template polynucleotide comprising periodic sequences, which can be random or predetermined sequences comprising not more than three different types of nucleotides; a primer which is complementary to a portion of a template polynucleotide, for example a 3′ portion of a template polynucleotide, or to a portion of a vector comprising a template (for example, a sequencing primer, discussed below); a set of nucleoside triphosphates; and a terminator nucleoside triphosphate. In various embodiments, the terminating nucleoside triphosphate also comprises a label. The primer oligonucleotide can also comprise a label. One or more nucleoside triphosphates can also comprise a label. The contacting occurs under conditions for a primer extension reaction to occur in the presence of a 3′ terminator. In the reaction, the primer oligonucleotide can hybridize to a portion of a template polynucleotide, for example a 3′ portion of a template polynucleotide, and can elongate to produce a nested set of elongation products, each of which terminates with the nucleotide comprising the terminator nucleoside triphosphate (Sanger F., et al., Proc. Natl. Acad. Sci. USA 76: 5463-5467, 1977). Because of the presence of the complement of the terminator nucleoside triphosphate (i.e., a “terminator complement”) at locations that can be evenly spaced locations, the nested set can appear as a ladder of evenly spaced nucleic acid fragments when detected following separation according to size. Separation according to size can be by standard methods, for example gel electrophoresis, capillary electrophoresis, or column chromatography. Detection of size-separated nucleic acid fragments can be by standard methods known in the art, in non-limiting examples, laser illumination of a fluorophore, or exposure to x-ray film of a radiolabeled sample.

In various embodiments of the invention, the sequence of the template oligonucleotide can be selected to be suitable for generation of a ladder. In some embodiments, a template oligonucleotide suitable for generation of a ladder comprises a periodic sequence comprising not more than three different types of nucleotides, plus a “terminator complement” type of nucleotide that is different from each of the not more than three different types of nucleotides. The terminator complement type of nucleotide is absent from the sequence of the template oligonucleotide except at one or more pre-determined positions. The remaining not more than three types of nucleotides occupy positions not occupied by the terminator complement type of nucleotide. A sequence of a template oligonucleotide can be of any sequence that does not cause an electrophoretic mobility anomaly in a standard separation medium, such as, for example, agarose, polyacrylamide, or a polymer used in capillary electrophoresis. In various embodiments, the template polynucleotide can comprise periodic sequences, for example, sequences of from about 5 to about 50 contiguous nucleotides, none of which are terminator complement type of nucleotide. In some embodiments, the periodic sequences can be about 20 nucleotides in length, about 25 nucleotides in length, or about 50 nucleotides in length. In some embodiments, the periodic sequences can contain from about 6 to about 20 contiguous nucleotides. In some embodiments, the periodic sequences do not have to be identical to one another. In various embodiments, a template polynucleotide can consist of stretches of a fixed number of contiguous nucleotides, for example nine contiguous nucleotides, each consisting of not more than three different types of nucleotides (for example, adenosine, thymidine and cytidine), plus a terminator complement type of nucleotide that differs from each of the not more than three different types of nucleotides (for example, guanidine). For example, the terminator complement type of nucleotide can be located, at intervals of ten nucleotides. For example, a polynucleotide of 200 nucleotides wherein every tenth nucleotide is a guanidine, will have 20 evenly-spaced guanidines. If this polynucleotide is used as a template in a standard polymerase-catalyzed sequencing reaction using as a terminator a non-extendable nucleoside triphosphate that is complementary to the terminator complement nucleotide, (for example a dideoxycytidine triphosphate if the terminator complement nucleotide is a guanidine), a ladder can be generated wherein fragments can differ in length by ten nucleotides.

In some embodiments, a template sequence can comprise two different terminator complement types of nucleotides. Different ladders can be generated from such a template sequence, by use of different terminator nucleoside triphosphates. For example, a template sequence can be used wherein cytidines and thymidines occupy alternating periodically spaced positions. In non-limiting example, a cytidine can occupy the 5^{th}, 15^{th}, and 25^{th} positions in a template sequence, whereas a thymidine can occupy the 10^{th}, 20^{th} and 30^{th} positions in the template sequence. Sequencing-type reactions using, for example, either a dideoxyGTP or a dideoxyATP as a terminator will yield size standard ladders comprising fragments of 5, 15, and 25 nucleotides in length or 10, 20, and 30 nucleotides in length, respectively.

In various embodiments, a template oligonucleotide suitable for generation of a ladder comprises a periodic sequence comprising not more than two different types of nucleotides, plus a “terminator complement” type of nucleotide that is different from each of the not more than two different types of nucleotides. For example, a template oligonucleotide not containing and guanidine nucleotides can have the sequence ACTTTCACTTTCACCCCCA (SEQ ID NO: 9). A ladder produced enzymatically using a ten nucleotide primer, this sequence as template, and a dideoxythymidine terminator would comprise evenly spaced fragments of 11, 17, 23, and 29 nucleotides.

In various embodiments, a template oligonucleotide suitable for generation of a ladder comprises a periodic sequence comprising not more than one type of nucleotide, plus a “terminator complement” type of nucleotide that is different from the one type of nucleotide. For example, a template oligonucleotide not containing and guanidine nucleotides can have the sequence ACCCCCACCCCCACCCCCA (SEQ ID NO: 10). A ladder produced enzymatically using a ten nucleotide primer, this sequence as template, and a dideoxythymidine terminator would comprise evenly spaced fragments of 11, 17, 23, and 29 nucleotides.

In various embodiments, methods are disclosed for generating a collection of single-stranded nucleic acid size standards that further comprises periodic sequences containing one or more “landmark” nucleic acids. A landmark can be particular useful in the automated analysis of nucleic acid samples subjected to separation by size, in that computer methods can be used to recognize landmarks and thereby determine the sizes of other size standard fragments as well as analyte fragments. Landmarks can also be useful to an investigator for aiding in the determination of the size of analytes or ladder fragments. The template polynucleotide can include adjacent periodic sequences of the same length containing not more than three different types of nucleotides separated by a nucleotide complementary to the terminator, as well as adjacent periodic sequences of differing length containing not more than three different types of nucleotides separated by a nucleotide complementary to the terminator. A periodic sequence that contains a nucleotide complementary to the terminator at locations differing in this way, i.e., a “landmark nucleotide,” gives rise to a detectable landmark nucleic acid when the polynucleotide is used as a template for generating nucleic fragments in the presence of a terminator. Following the above example, in a template polynucleotide of 200 bases in which every tenth base is a guanidine, the 48^{th} and 52^{nd} bases can also each be a guanidine. A ladder generated using this template and a terminator comprising a cytidine will include periodic sequences containing detectable landmarks consisting of three fragments differing in length by two bases. Following electrophoretic separation, these landmarks can be readily identifiable as a “triplet” of closely spaced fragments as distinct from other fragments which differ in length by intervals of 10 bases (and hence can appear further apart if visualized following electrophoretic separation).

In various embodiments, a template polynucleotide can be at least about 200 nucleotides in length, at least about 500 nucleotides in length, or at least about 1000 nucleotides in length, and the method can be used to generate size standards that can range in size from about 10 contiguous nucleotides or 25 contiguous nucleotides in length to at least about 200 contiguous nucleotides in length, to at least about 500 contiguous nucleotides in length, or to at least about 1000 contiguous nucleotides in length. In various embodiments, the method can be used to generate size standards ranging from 500 contiguous nucleotides to 1000 contiguous nucleotides in length. In various embodiments, implementation of the method can generate at least about 10 size standard polynucleotides, at least about 20 size standard polynucleotides, at least about 30 size standard polynucleotides, at least about 40 size standard polynucleotides, or at least about 50 size standard polynucleotides.

Generation of a size standard template can be by any suitable technique of nucleic acid synthesis. For example, a polymerase extension reaction method can be used, as described in copending application docket No. 9692-000013, entitled “Method Of Generating Long Nucleic Acid Molecules Of Defined Sequence” by Chu-an Chang et al., filed Jan. 15, 2003, which is hereby incorporated by reference in its entirety. For example, a template oligonucleotide described in certain embodiments in copending application docket No. 9692-000013 can be used as a template to generate a size standard template. Copending application docket No. 9692-000013 also discloses, in certain embodiments, uses of templates described in that application. Well known organic chemical synthesis methods can also be used to generate a size standard template, for example, automated methods utilizing 3′-phosphoramidites (Horvath et al., Methods Enzymol. 154: 314-326, 1987). In addition, a size standard template can be propagated in a vector (as described below), thereby providing a cloning method for production of the template.

In other embodiments, a template sequence can be designed to include restriction sites. Inclusion of a restriction site facilitates cloning of the template polynucleotide in a vector. In various embodiments, the method comprises forming a recombinant vector comprising a DNA polynucleotide. In some embodiments, the parent vector of the recombinant vector can be a bacteriophage or a plasmid. The parent vector is preferably a vector that is suitable for sequencing of an insert (Sanger F., et al., Proc. Natl. Acad. Sci. USA 76: 5463-5467, 1977). When this method is used, only the complement of the terminator complement nucleotide needs to be used as a terminator. In these embodiments, routine methods (Sambrook, J., et al., Molecular Cloning: A Laboratory Manual; Cold Spring Harbor Laboratory: Plainview, N.Y., 1989) can be used to generate a vector comprising the polynucleotide. In various embodiments, the method comprises inserting the polynucleotide in single-stranded or double-stranded form into a vector. The polynucleotide can be inserted by ligation. Ligation can be blunt-end ligation. In an alternative embodiment, “directional cloning” can be used to form a recombinant vector. In these embodiments, the sequence of a polynucleotide for generating a ladder can comprise one or more restriction sites, which can be at predetermined locations. The restriction sites can be selected to provide, upon cleavage by a restriction enzyme, DNA fragment termini that are compatible with termini available in a parent vector.

The size standard nucleic acids produced by the methods described herein can further comprise a label or reporter group. In non-limiting example, the label or reporter group can be a fluorophore such as VIC®, FAM®, ROX®, LIZ® or TAMRA®) (Applied Biosystems, Inc.), a chromophore, a biotin, a hapten (for example bromodeoxyuridine or digoxygenin), a chemiluminescent moiety, a radioisotope (for example, a ³H, a ¹⁴C, a ³²p, or a ³³P), or a spin label. The label can be introduced by any method known in the art. For example, a terminator nucleoside triphosphate used in the invention can comprise a fluorescently tagged dideoxynucleotide triphosphate chain terminator. The label or reporter group can also be introduced by incorporation or covalent attachment to a primer oligonucleotide, or incorporation or covalent attachment to a deoxyribonucleoside triphosphate or a ribonucleoside triphosphate. In various embodiments, the label or reporter group can be a fluorophore moiety covalently attached to a terminator nucleoside triphosphate. In some embodiments, a fluorophore can have excitation and/or emission wavelengths which are distinguishable from dyes that are commonly used in sequencing reactions, for example, the fluorophores used in “BigDye” terminator kits (Applied Biosystems, Inc.) When such a fluorophore is used, a ladder produced using the invention can be added to a sample of the reaction products of a sequencing reaction. The ladder thereby can provide an internal size standard. In some embodiments, the internal size standards can be detected using an automated sequence analysis system, for example a PRISM® 377 DNA Sequencer (Applied Biosystems, Inc.) The use of internal size standards can be used by an investigator to aid in the accurate manual measurement of the size of an analyte DNA sample.

### EXAMPLE 1

This example illustrates generation of a set of fragments that can be a ladder uniformly-spaced in size. In this example, a DNA fragment comprising the sequence 5′-GCTACTACTAGCTACTACTAGCTACTACTAGTCTA-3′ (SEQ ID NO: 1) can be inserted into a restriction site of a vector of known sequence, for example the Hind III site of a pUC 18 plasmid (Yanisch-Perron et al., Gene 33: 103-119, 1985). A sequencing primer complementary to the vector adjacent to the insertion site of the fragment, for example GTAAAACGACGGCCAGT (SEQ ID NO: 2) (New England Biolabs, Inc.) can then be utilized as a primer for a DNA polymerase-catalyzed synthesis reaction in a mixture comprising a labeled dideoxycytidine as a terminator. As a result of elongation, a nested set of reaction products would be produced as shown in table 1:

TABLE 1

| | | |
|---|---|---|
| | | Length |
| Sequence | SEQ ID NO: | (bases) |

| | | |
|---|---|---|
| GTAAAACGACGGCCAGTGC* | SEQ ID NO:3 | 19 |
| GTAAAACGACGGCCAGTGCC* | SEQ ID NO:4 | 20 |
| GTAAAACGACGGCCAGTGCCATAGAC* | SEQ ID NO:5 | 26 |
| GTAAAACGACGGCCAGTGCCATAGACTAGTA | SEQ ID NO:6 | 36 |
| GTAGC* | | |
| GTAAAACGACGGCCAGTGCCATAGACTAGTA | SEQ ID NO:7 | 46 |
| GTAGCTAGTAGTAGC* | | |
| GTAAAACGACGGCCAGTGCCATAGACTAGTA | SEQ ID NO:8 | 56 |
| GTAGCTAGTAGTAGCTAGTAGTAGC* | | |

wherein C* represents a labeled dideoxycytidine.
The reaction products resulting from the synthesis reaction differ from one another by intervals of 10 nucleotides (SEQ ID NO: 5 through SEQ ID NO: 8) when the DNA fragment (SEQ ID NO: 1) serves as template. Separation of these fragments according to size using a standard separation method, for example gel electrophoresis or capillary electrophoresis, would provide a ladder of DNA markers spaced apart by an interval of 10 bases. Because the length of the sequences would be known, the ladder could be used for determining the size of an analyte DNA which was subjected to identical separation conditions.

### EXAMPLE 2

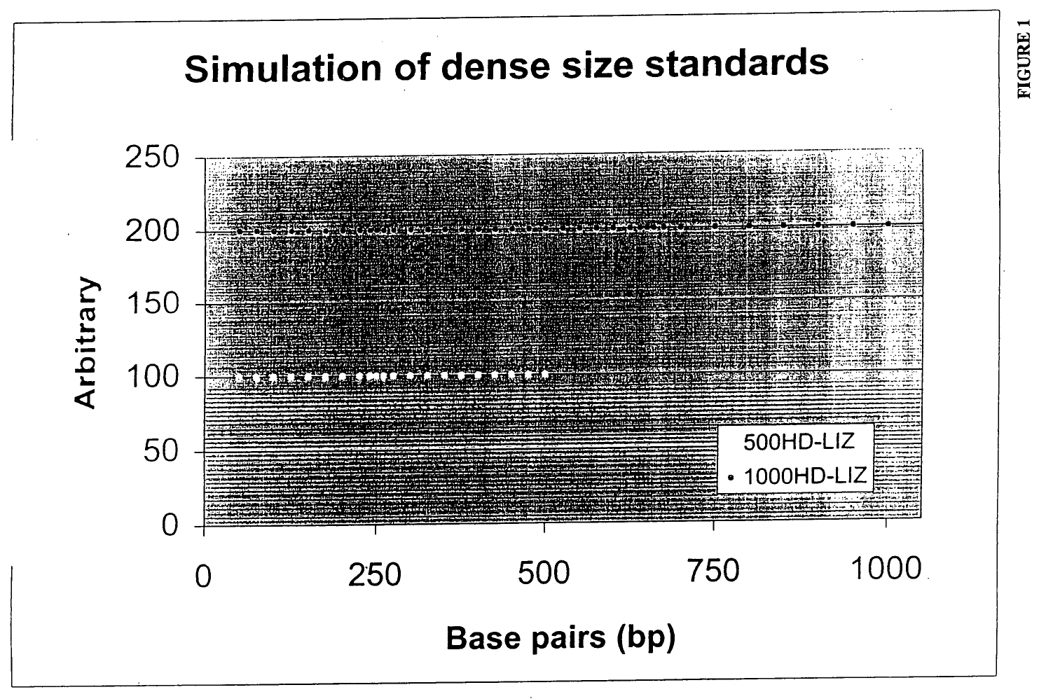

This example illustrates a simulation of expected positions of DNA size standards following electrophoretic separation using capillary electrophoresis. As shown in FIG. 1, in the upper row, dark spots represent an expected distribution of a ladder comprising markers up to 1000 nucleotides in length. In the lower row, light spots represent an expected distribution of a ladder comprising markers up to 500 nucleotides in length. Expected positions for landmarks around a comparatively short (approx. 250 nucleotide) size marker and a comparatively long (approximately 650 nucleotide) size marker are also shown.

As various changes could be made in the above methods and compositions without departing from the scope of the invention, it is intended that all matter contained in the above description be interpreted as illustrative and not in a limiting sense.

All references cited in this specification are hereby incorporated by reference in their entirety. The discussion of the references herein is intended merely to summarize the assertions made by their authors and no admission is made that any reference constitutes prior art relevant to patentability. Applicant reserves the right to challenge the accuracy and pertinency of the cited references.

1. A method for generating nucleic acid size standards, the method comprising:
providing a template polynucleotide which comprises periodic sequences of from about 5 to about 50 contiguous nucleotides containing not more than three different types of nucleotides and wherein adjacent periodic sequences are separated by a terminator complement type of nucleotide different from each of the not more than three different types of nucleotides; and
performing a primer extension reaction on the template polynucleotide in the presence of a primer which is sufficiently complementary to the template polynucleotide to hybridize therewith, a nucleic acid polymerase, nucleoside triphosphate molecules suitable for a polymerase extension of the primer on the template polynucleotide and a 3′ terminating nucleoside triphosphate which is complementary to the terminator complement type of nucleotide.

2. A method according to claim 1 wherein the template polynucleotide is an artificial sequence.

3. A method according to claim 1 wherein the periodic sequences are substantially of the same length.

4. A method according to claim 3 wherein the periodic sequences are about 25 nucleotides in length.

5. A method according to claim 1 wherein the periodic sequences contain from about 6 to about 20 contiguous nucleotides.

6. A method according to claim 1 wherein the template polynucleotide is at least about 500 nucleotides in length and the method generates size standards which are from about 25 contiguous nucleotides to at least about 500 contiguous nucleotides in length.

7. A method according to claim 6 wherein about 20 size standard fragments are generated.

8. A method according to claim 6 wherein the template polynucleotide is about 1000 nucleotides in length and the method generates size standards which are from about 25 contiguous nucleotides to about 1000 contiguous nucleotides in length.

9. A method according to claim 8 wherein about 40 size standard fragments are generated.

10. A method according to claim 1 wherein the 3′ terminating nucleoside triphosphate is a dideoxynucleoside triphosphate or a 3′ amino nucleoside triphosphate.

11. A method according to claim 1 wherein the 3′ terminating nucleoside triphosphate further comprises a covalently attached label.

12. A method according to claim 11 wherein the label is a fluorophore, a chromophore, a biotin, a hapten, a radioisotope, a chemiluminescent moiety, or a spin label.

13. A method according to claim 12 wherein the label is a fluorophore selected from the group consisting of VIC, FAM, ROX, LIZ and TAMRA.

14. A method according to claim 12 wherein the label is a radioisotope selected from the group consisting of ³H, ¹⁴C, ³²p, and ³³P.

15. A method according to claim 1 wherein some of the periodic sequences contain one or more landmark nucleotides.

16. A method according to claim 15 wherein the one or more landmark nucleotides is a triplet of nucleotides comprising the identical terminator complement type of nucleotide.

17. A method according to claim 1 wherein the template polynucleotide contains restriction sites suitable for cloning the template.

18. A method according to claim 1 wherein the periodic sequences are random sequences of the not more than three different types of nucleotides.

19. A method according to claim 18 wherein the not more than three different types of nucleotides comprise not more than two different types of nucleotides.

20. A method according to claim 19 wherein the not more than two different types of nucleotides comprise not more than one type of nucleotide.

21. A method according to claim 1 wherein the primer further comprises a covalently attached label.

22. A method according to claim 21 wherein the label is a fluorophore, a chromophore, a biotin, a hapten, a radioisotope, a chemiluminescent moiety, or a spin label.

23. A method according to claim 22 wherein the label is a fluorophore selected from the group consisting of VIC, FAM, ROX, LIZ and TAMRA.

24. A method according to claim 23 wherein the label is a radioisotope selected from the group consisting of ³H, ¹⁴C, ³²p, and ³³P.

25. A method for generating nucleic acid size standards, the method comprising:
combining in a mixture, a template polynucleotide having a 5′ portion and a 3′ template portion, a primer which is sufficiently complementary to the 3′ portion of the polynucleotide to hybridize therewith, a nucleic acid polymerase, nucleoside triphosphate molecules suitable for a polymerase extension of the primer on the template polynucleotide and a 3′ terminating nucleoside triphosphate; and
maintaining the mixture under conditions suitable for a primer extension reaction, wherein the template polynucleotide comprises periodic sequences of from about 5 to about 50 contiguous nucleotides none of which are complements to the 3′ terminating nucleoside triphosphate and wherein adjacent periodic sequences are separated by the complementary nucleotide of the 3′ terminating nucleoside triphosphate.

26. A method according to claim 25 wherein the template polynucleotide is an artificial sequence.

27. A method according to claim 25 wherein the periodic sequences are substantially of the same length.

28. A method according to claim 27 wherein the periodic sequences are about 25 nucleotides in length.

29. A method according to claim 25 wherein the periodic sequences contain from about 6 to about 20 contiguous nucleotides.

30. A method according to claim 25 wherein the template polynucleotide is at least about 500 nucleotides in length and the method generates size standards which are from about 25 contiguous nucleotides to at least about 500 contiguous nucleotides in length.

31. A method according to claim 30 wherein about 20 size standard fragments are generated.

32. A method according to claim 30 wherein the template polynucleotide is about 1000 nucleotides in length and the method generates size standards which are from about 25 contiguous nucleotides to about 1000 contiguous nucleotides in length.

33. A method according to claim 32 wherein about 40 size standard fragments are generated.

34. A method according to claim 25 wherein the 3′ terminating nucleoside triphosphate is a dideoxynucleoside triphosphate or a 3′ amino nucleoside triphosphate.

35. A method according to claim 25 wherein the 3′ terminating nucleoside triphosphate further comprises a covalently attached label.

36. A method according to claim 35 wherein the label is a fluorophore, a chromophore, a biotin, a hapten, a radioisotope, a chemiluminescent moiety, or a spin label.

37. A method according to claim 36 wherein the label is a fluorophore selected from the group consisting of VIC, FAM, ROX, LIZ and TAMRA.

38. A method according to claim 36 wherein the label is a radioisotope selected from the group consisting of ³H, ¹⁴C, ³²p, and ³³P.

39. A method according to claim 25 wherein some of the periodic sequences contain one or more landmark nucleotides.

40. A method according to claim 39 wherein the one or more landmark nucleotides is a triplet of nucleotides comprising the identical type of terminator nucleotide.

41. A method according to claim 25 wherein the template polynucleotide contains restriction sites suitable for cloning the template.

42. A method according to claim 25 wherein the periodic sequences are random sequences of not more than three types of nucleotides.

43. A method according to claim 42 wherein the not more than three different types of nucleotides comprise not more than two different types of nucleotides.

44. A method according to claim 43 wherein the not more than two different types of nucleotides comprise not more than one type of nucleotide.

45. A method according to claim 25 wherein the primer further comprises a covalently attached label.

46. A method according to claim 45 wherein the label is a fluorophore, a chromophore, a biotin, a hapten, a radioisotope, a chemiluminescent moiety, or a spin label.

47. A method according to claim 46 wherein the label is a fluorophore selected from the group consisting of VIC, FAM, ROX, LIZ and TAMRA.

48. A method according to claim 46 wherein the label is a radioisotope selected from the group consisting of ³H, ¹⁴C, ³²p, and ³³P.
